(19) United States
(12) Patent Application Publication
(10) Pub. No.: US 2003/0003059 A1
(43) Pub. Date: Jan. 2, 2003

(54) Dentifrice compositions

(76) Inventor: Frederic Dana, Deauville Cedex (FR)

Correspondence Address: COLEMAN SUDOL SAPONE, P.C., 714 Colorado Avenue, Bridgeport, CT, 06605-1601, US

(21) Appl. No.: 10/004,111

(22) Filed: Nov. 15, 2001

Related U.S. Application Data
(63) Continuation-in-part of application No. 09/872,829, filed on Jun. 1, 2001, now abandoned.

(30) Foreign Application Priority Data
Apr. 3, 2001 (FR) 0104614

(51) Int. Cl. A61K 7/16

(52) U.S. Cl. 424/49

(57) ABSTRACT

The present invention relates to oral care compositions which provide a means to deliver actives which are useful in the prevention, treatment and/or management of dental and related tissue conditions, including dental caries, dental cavities, microbial flora, tartar, periodontal and related gum disease. In addition, the present invention may be used in the healthy maintenance of teeth and gums. Further aspects of the present invention are related to the present compositions being useful to whiten teeth and otherwise favorably impact the cosmetic appeal of the teeth and gums of a subject or patient. A further aspect of the present invention is directed to the inclusion of effective amounts of colostrum in dental care compositions for the unexpectedly high efficacy such formulations provide in inhibiting, reducing or otherwise preventing microbial growth, dental caries, plaque, cavities and gum disease, including periodontal disease. The use of colustrum with other enzymes as otherwise described herein represents a particularly preferred embodiment for use in the present invention because of the unexpected antimicrobial activity exhibited by the enzyme combination.

### RELATED APPLICATIONS

This application is a continuation-in-part application of Ser. No. 09/872,829 of same title, filed Jun. 1, 2001.

### FIELD OF THE INVENTION

The present invention relates to oral care compositions which provide a means to deliver actives which are useful in the prevention, treatment and/or management of dental and related tissue conditions, including dental caries, dental cavities, microbial flora, tartar, periodontal and related gum disease. In addition, the present invention may be used in the healthy maintenance of teeth and gums. Further aspects of the present invention are related to the present compositions being useful to whiten teeth and otherwise favorably impact the cosmetic appeal of the teeth and gums of a subject or patient.

A further aspect of the present invention is directed to the inclusion of effective amounts of colostrum in dental care compositions for the unexpectedly high efficacy such formulations provide in inhibiting, reducing or otherwise preventing microbial growth, dental caries, plaque, cavities and gum disease. The use of colustrum with other enzymes as otherwise described herein represent particularly preferred additives for use in because of the unexpected and synergistic (i.e., more than additive) antimicrobial activity exhibited by the enzyme combination.

Methods of treating the teeth, mouth and/or gum tissue of mammals, and in particular, domesticated animals and human children with compositions according to the present invention are also aspects of the present invention.

### BACKGROUND OF THE INVENTION

The thickening of dental plaque from bacteria is primarily due to the nutritive supply of sucrose, because bacteria all have enzymes for glycolysis. In this manner, the analysis of dental plaque shows proteins that have an adhesive characteristic, as well as polysaccharides, produced by the bacterial metabolism of sucrose, and which form the base material of the plaque matrix.

There is a difference, however, between extra-cellular polysaccharides:

a) soluble

dextrans: glucose polymers that form linear chains α-1.6 and multiple branches α-1.3, and sometimes α-2.1.

leavens: polymers that are linear and sometimes branched by fructose. The osidic link is type β-2.6 and β-2. 1.

glucans: glucose homopolymers.

b) insoluble

mutanes: glucose polymers α-1.3 and sometimes α-1.6.

And intrabacterial polysaccharides:

amylopectin: linear polymers of glucose that form chains α-1.4 and branches in α-0.6

In addition, we also know that the compounds used for toothpaste are currently most often presented in the form of a paste, a solution or a powder. To use them, it is necessary to measure a specific quantity and apply it to the teeth using a toothbrush. Use of this type of brush is a constraint for multiple reasons.

Among other reasons, the need to use a toothbrush is a reason why it is difficult to get children to brush their teeth regularly. Likewise, when one is out of the house, it is impossible to clean one's teeth after a meal or before an exam. It is also very difficult to clean the teeth of animals, especially dogs.

For a long time prior to the present invention, the process of adding substances to dental care compositions such as toothpaste, mouthwash, dental floss, sprays, chewing gums and tablets, which are designed as prophylactic or therapeutic agents for the treatment of teeth, gums or saliva has been common practice.

There exists in the art dental products which contain various fluorides as active substances for the prevention of dental caries and cavities, and active organic and biochemical substances which prevent or reduce the formation of dental plaque and/or tartar. The primary drawback of each of these substances, and the dental products in the prior art, is that the active substances do not have enough time to remain in contact with the teeth, gums and saliva to become fully activated, especially due to the relatively short time period a composition may be exposed to the teeth during brushing or using an oral rinse, mouthwash or other dental product of the art. In such products, a significant portion of the potential effectiveness of the products is lost. This is especially true for children where compliance using traditional products is marginal at best.

Also, there exists a long-felt need for enhanced dental hygiene in dogs, cats and other domestic animals, which presently is non-existent, due to the impossibility of effectively brushing the teeth of domestic animals and the short application time of ancillary dental products such as sprays, pastes, chewable products and tablets.

Dental caries is an infectious disease which is ubiquitous in humans. Dental caries is caused by colonies of bacterial microorganisms and in particular Actinomyces sp. and Streptococcus mucans, which form on the surfaces of the teeth to form a very adhesive dental plaque.

### OBJECTS OF THE INVENTION

It is an object of the invention to provide dental compositions which may be used to deliver active agents to the surface of the teeth of mammals, especially domestic animals such as dogs and cats and humans, particularly human children.

It is another object of the present invention to provide compositions which contain ingredients which maximize the surface and duration of coverage of the compositions on the teeth of the subjects in order to enhance delivery of active agents for preventing and/or treating dental diseases and/or conditions and reducing microbial flora, tartar, plaque and the like.

It is still another object of the present invention to provide additives which may be included in dental care composition to enhance the anti-caries, anti-plaque and anti-microbial activity of the compositions.

It is yet another object of the present invention to provide methods for treating and/or preventing dental conditions and/or disease states including dental caries, tartar, plaque and the like or for reducing microbial flora which may be responsible for dental caries.

Any one or more of these and/or other objects of the present invention may be readily gleaned from a review of the description of the invention which follows.

### SUMMARY OF THE INVENTION

In one aspect, the present invention provides a new dental or oral care composition which is gelled or may form a gelled matrix after hydration with an aqueous solution such as saliva and finds particular use in oral hygiene, for the treatment of teeth and gums. The oral care compositions according to the present invention include an effective amount of a gelling agent comprising about 0.05% to about 25% by weight of said oral care composition, preferably about 0.1% to about 20% by weight and more preferably about 0.5% to about 15% by weight of said composition, said gelling agent comprising silica (micronized, hydrated or aerosol) as a lipophilic gelling agent, alone or in combination with at least one other orally compatible lipophilic gelling agent or preferably, a hydrophilic gelling agent. In preferred embodiments according to the present invention, the gelling agent according to the present invention comprises a mixture of micronized, hydrated or aerosol silica and a hydrophilic gelling agent, with the weight ratio of silica to hydrophilic gelling agent in the final dental care composition falling within the range of about 1:1 to about 1:6, more preferably about 1:1 to about 1:3, such composition being specifically adapted to adhere to teeth and provide a sustained or prolonged release action by the ability of the composition to adhere to teeth.

In a second aspect of the present invention, dental care compositions according to the present invention comprise colostrum and optionally, at least one additional antimicrobial enzyme, preferably at least one enzyme selected from the group consisting of lactoperoxidase and lysozyme, in addition to other optional agents for delivery to the teeth and oral cavity are also contemplated. Dental care compositions according to this aspect of the present invention may be used alone, or optionally, in combination with the first aspect of the invention, which relates to the inclusion of a gelling agent to produce a gelled matrix. Compositions according to this aspect of the present invention are particularly useful for the prevention, reduction or elimination of dental caries, cavities, plaque, microbial growth and gum diseases, including periodontal disease because of the unexpected and exceptional antimicrobial activity that such compositions display. In alternative embodiments of this aspect of the present invention, optional proteins selected from the group consisting of lactoferrin, dextranase, mutanase, cellulase, amylo-glucosidase, papain, bromelin and mixtures thereof, all included in effective amounts may be added to the dental care compositions according to the present invention.

The present invention may be used in oral care products in mammals, especially humans, including children and domestic animals by providing controlled doses (in certain cases, controlled or sustained release doses) of elements which have a specific activity as well as extended action of those elements. The present compositions may be used effectively in the fight against dental cavities, against microbial flora and to combat hard or dental plaque by coating the anterior and posterior sides of the teeth and by fighting and/or removing tartar and/or calcium deposits encrusted between the teeth.

In this aspect of the present invention, the present compositions permit active substances such as fluorides, chemical, biochemical or biological antimicrobial agents, filmogen agents, anti-tartar agents, vitamins, minerals, micronutrients and other nutrients for the teeth, such as calcium, for example, to be applied to the surface of the teeth and to keep them there for an extended period of time so that their properties may be activated and exhibit maximum effect.

As a result of the adhesive characteristics of dental plaque, its mode or type of composition and the hygienic products that are too rapidly put into contact with the teeth or gums, and in addition, that are not available in an individual format, it has been discovered that a remedy for these inconveniences is a dental care product according to the present invention that is available in tablet form and gels when it comes into contact with water or other aqueous solution, notably saliva, or a semi-solid gel that has been partially or fully pre-gelled into a single dose to be applied directly onto the teeth.

This tablet or semi-solid gel contains in its gelling matrix active substances, which may be hydrophilic or lipophilic, preferably agents which are hydrophilic and agents which are lipophilic which would act directly on dental plaque and bacteria to remove, eliminate, reduce and/or inhibit the growth of bacteria or strengthen teeth and provide dental care. The present compositions are designed to be applied directly onto the teeth and gums, and to remain in contact with the teeth for a period of time sufficient to release the active elements onto the teeth, gum tissue and where applicable, into the saliva. The release of active from the matrix according to the present invention onto the teeth is therefore gradual and sustained by the flow of saliva; that is to say, the active hydrophilic and lipophilic substances are “kept by retaining them in a reservoir.” In this manner, one can achieve optimal use of the full action of the active materials in order to offer a significant contribution to the healthy maintenance of teeth and gums anywhere and at any time, without brushing or rinsing, in a single dose format.

Without being limited by way of theory, it is believed that the mechanism of activating the product of this invention comprises placing the dry or partially hydrated dental composition into the mouth, allowing the composition, where applicable, to hydrate to produce a gel material (in other embodiments, the material will be at least partially pre-gelled and can be applied directly to the teeth without a first hydration step), at least partially spreading the gel matrix onto or over the teeth, and then allowing active substances within the gelled matrix to be gradually released from the matrix so that the actives will exhibit maximum effect on the teeth or gum tissue. After the matrix of the product of the invention gels, generally after contact with saliva during the chewing process, the active substances that are trapped in this matrix are then gradually released by saliva and chewing, by the mechanical effect of repeated friction from the silica or any abrasive material, as well as by the passage of the tongue over the teeth as a brush.

Compositions according to the present invention in any aspect or embodiment may be formulated for oral delivery advantageously in the form of a toothpaste, gel, paste, pill, tablet, especially including a chewable tablet, dry tablet, buccal tablet, spray, solution, lozenge, soaked napkin, dental floss or any other prepared form applicable to ingestible dental hygiene products in a single-dose form

### DETAILED DESCRIPTION OF THE INVENTION

The following terms shall be used throughout the present specification to describe the invention. It is noted here that where a term has not been given a specific definition in the specification, the term will have that meaning which is understood by those of skill within the applicable art.

The term “patient” or “subject” shall mean a mammal, preferably a domesticated mammal or human, preferably including a human child having a need for dental care because that animal has dental plaque, tartar, microbial flora, dental caries or cavities, halitosis, gum disease or other dental disease or condition or otherwise wishes to make the teeth less sensitive, cosmetically treat the teeth in order to whiten the teeth or to prevent or slow the aging process on dental tissue.

The term “long-term” is used throughout the specification to describe the period that a gel or film will remain in contact with the teeth or gums of the subject or patient after initial contact. In the present invention, the gel and/or the film formed from the gel will remain on the teeth for a period of at least about 5 minutes, preferably at least about 30 minutes and more preferably at least about 1 hour or longer. In certain aspects of the present invention, the film produced by delivery to the teeth of a subject or patient will remain on the teeth until such time as the teeth are brushed.

The term “gelled” or “gel” is used throughout the specification to describe the condition of certain preferred embodiments of the dental care compositions according to the present invention. A gelled composition according to the present invention is a product in a gel-like state having a viscosity ranging from about 250 cps to about 10,000 cps, preferably about 500 cps to about 3,500 cps, even more preferably about 750 cps to about 1,500 cps. The gelled feature of certain embodiments of the present invention is an important feature inasmuch as it is the gelled composition which has a viscosity high enough to allow the formulation to adhere to or coat the teeth and/or gums of the subject or patient using the composition. From the adhered or coated composition, the active agents within the present compositions may be delivered to the teeth or gums of the subject in a manner consistent with sustained or maximum delivery of actives to the sites of activity on the teeth or gums.

The term “viscosity” is used to describe an important feature of certain embodiments according to the present invention. Viscosity is a property or quality of compositions according to the present invention which relates to the resistance of the composition to flow. For purposes of the present invention, viscosity is measured in centipoise units as determined by a Brookfield Programmable Viscometer using the required DV-II+spindle at 0.5 rpm. Compositions according to the present invention which have lower viscosities, i.e., viscosities which are less than about 1,000-1,500 centipoise (cps) units tend to be flowable. As the viscosity of the compositions decreases, the flowability of the composition increases. Compositions which have viscosities which are less than about 1,000-1,500 cps are readily flowable and deliverable to sites on the teeth and gums, whereas those compositions which have viscosities which are greater than about 1,500 cps tend to be less flowable, but still deliverable within the context of the present invention.

The term “effective” is used throughout the specification to describe amounts of specific chemicals or components which are included in dental care compositions according to the present invention. An effective amount of a specific chemical or component is that amount which is included to produce its intended effect in the final composition. For example, an effective amount of a gelling agent, is that amount of such an agent which will gel the final composition upon exposure to saliva or other aqueous solution, including water. In the case of an anti-tartar compound, the amount of such a compound will be that amount which instills anti-tartar characteristics to the final composition used in the mouth of the subject of patient.

The term “silica”, “hydrated silica”, “micronized silica”, “aerosol silica” is used throughout the specification to describe an additive used in the present invention which is derived from silicon dioxide and is added for the benefits it provides as a base material to provide a gel-like consistency to the dental care compositions as well as aiding in the continued release of actives from film which is in contact with the teeth and/or gums of the subject/patient after delivery of the composition to the teeth and/or gums. The silica used in the present invention is hydrophobic (lipophilic) in nature, is naturally occurring and is processed from a naturally occurring mineral (SiO₂) into granular or beaded form. It has the ability of absorbing lipophilic active agents, such as oils, lipophilic vitamins and other active agents. Preferred forms of silica for use in the present invention include micronized silica, aerosol type silica, amorpohous silica and hydrated silica, all of which forms of silica are well known in the art. In certain compositions the silica is most preferably both micronized and hydrated. These materials are included in compositions according to the present invention in amounts effective to facilitate gelling and/or the release of actives from the gel matrix which forms in contact with the teeth/gums of the subject or patient after administration of the present dental care compositions.

The term “lipophilic gelling agent” is used throughout the specification to describe certain gelling agents which may be used in the present invention and are lipophilic in character. These gelling agents include silica (also known as silica gel, as otherwise described above), as well as other pharmaceutically acceptable lipophilic gelling agents such as acrylic gelling agents, which are copolymers of (meth)acrylate monomers and other monomers such as (meth)acrylic acid and poly(meth)acrylate polymeric gelling agents such as those based on (meth)acrylic acid, methyl (meth)acrylate, ethyl (meth)acrylate, n-propyl (meth)acrylate, isopropyl (meth)acrylate, n-butyl (meth)acrylate, isobutyl (meth)acrylate, t-butyl (meth)acrylate, 2-ethylhexyl (meth)acrylate, isobomyl (meth)acrylate, cetyl (meth)acrylate, lauryl (meth)acrylate, stearyl (meth)acrylate, dodecyl (meth)acrylate, dimethylaminoethyl (meth)acrylate, (meth)acrylic diethylene glycol ethoxylate, 2-methoxyethyl (meth)acrylate, 3-methoxybutyl (meth)acrylate, phenoxyethyl (meth)acrylate, tetrahydrofurfuryl (meth)acrylate and cyclohexyl (meth)acrylate and polyvinylpyrrolidone polymers (PVP), among numerous others. Lipophilic gelling agents are useful for accommodating (solubilizing, emulsifying) lipophilic actives which are desirably added to compositions according to the present invention and to modify or alter the kinetics of release of hydrophilic compounds from the dental compositions. In certain aspects of the present invention any one or more of these lipophilic gelling agents may be substituted for the silica gelling agent in compositions according to the present invention. When used, these agents comprise about 0.05% to about 15% by weight of the final composition.

The term “hydrophilic gelling agent” is used to describe certain gelling agents which are advantageously added to silica or other lipophilic gelling agents in certain preferred embodiments according to the present invention to produce compositions which exhibit both a lipophilic character from the silica or other lipophilic gelling agent and a hydrophilic character from the hydrophilic gelling agent. Preferred hydrophilic gelling agents for use in the present invention include gelling agents which are orally compatible and ingestible and include for example, carboxymethyl cellulose, methyl cellulose, methylethyl cellulose, hydroxymethyl cellulose, hydroxyethyl cellulose, hydroxypropyl cellulose, hydroxypropylmethyl cellulose, guar gum (including the hydroxy guar derivative), carob gum, karaya gum, algin, carageenan, and mixtures thereof. In general, these hydrophilic gelling agents are included in final dental care compositions according to the present invention in amounts ranging from about 0.05% to about 25% by weight, more preferably about 0.1% to about 15% by weight.

Preferred hydrophilic gelling agents for use in the present invention are selected from the hydrophilic cellulose gelling agents, most preferably, carboxymethyl cellulose or carageenan or a mixture of a lactomannan (e.g., guar gum or carob gum), algin and carageenan.

In certain aspects of the present invention, compositions comprise a mixture of silica and at least one hydrophilic gelling agent in a weight ratio of silica to hydrophilic gelling agent of about 1:1 to about 1:6, with a preferred weight ratio being about 1:1 to about 1:3. Other lipophilic gelling agents may also be added to the present compositions, in amounts generally ranging from about 0.1% to about 15% by weight, more preferably about 0.2% to about 5% by weight.

Compositions according to the present invention are characterized in that they comprise a base material in powder form that has already absorbed various active hydrosoluble or lipophilic elements or materials; this base material constitutes the future matrix, as it is made up of organic and/or mineral matter that gels rapidly upon contact with water and is easily digestible, as it may be bitten, chewed, licked or directly applied, for human or animal applications.

Compositions according to the present invention comprise an effective amount of silica in an amount ranging from about 0.05% to about 25% by weight, preferably about 0.1% to about 15% by weight, optionally, in combination with at least one digestible hydrophilic gelling agent selected from the group consisting of a hydrophilic cellulose gelling agent, guar gum, carob gum, karaya gum, algin, carageenan and mixture thereof, said silica and said hydrophilic gelling agent being included in said dental care composition in preferred weight ratios of silica to hydrophilic gelling agent within the range of about 1:1 to about 1:6. Optionally, additional lipophilic gelling agents may be added to the silica alone or in combination with the hydrophilic gelling agents in amounts ranging from about 0.05% to about 15% by weight of the final compositions, more preferably about 0.5% to about 10% by weight. When combinations of gelling agents (which gelling agents include silica, hydrophilic gelling agents and optionally, other lipophilic gelling agents) are used in the present invention, the amount of silica which is included in the mixture of gelling agents ranges from about 15% to about 45% by weight of the gelling agent mixture and the hydrophilic gelling agent ranges from about 40% to about 85% by weight of the gelling agent mixture, the remainder of the gelling agent comprising a lipophilic gelling agent as otherwise described herein.

In addition to silica and optionally, a hydrophilic gelling agent, compositions according to the present invention comprise at least one additional agent selected from the group consisting of polyalcohol (polyol) based diluents (non-cariogenic polyalcohols) including polyols such as sorbitol, xylitol, mannitol, maltitol, lactitol, glycerol (glycerine) and propylene glycol, among others, in amounts ranging from about 0.1% to about 85% by weight. Although the amount of polyol diluent which may be used in the present compositions may range widely over a broad range, when silica is used alone (i.e., without the inclusion of a hydrophilic gelling agent), the preferred amount of diluent ranges from about 5% to as much as 65% by weight of the final composition.

Other additives which may be included in the present compositions are selected from the group consisting of anti-cavity agents, anti-microbial agents, anti-tartar agents, immune agents, biological anti-bacterial agents, biochemical anti-plaque agents, film-forming agents, dental enamel mineralizers, dental enamel protectors and whiteners, sweeteners, agents to improve appearance, fine abrasives, sweeteners, anti-halitosis agents, anti-inflammatory agents, ingredients to reduce sensitivity of teeth, coloring agents, sweeteners, especially non-nutritive sweeteners, flavoring agents, vitamins (especially including vitamins A, D and E for children) and nutritive cleaners, anti-aging compositions and mixtures thereof, all of which may be included in the present compositions in effective amounts. One of ordinary skill will be able to readily include individual additives in effective amounts for their expected activity in the treatment of teeth and gums of a subject or patient.

Compositions according to the present invention, by modifying the formulations using the above-described additives, may be useful for the treatment or prevention of dental caries, as a sensitive teeth formulation, as an anti-plaque or anti-tartar composition, as a composition to prevent halitosis, as a dental composition to promote the whitening of teeth, as an anti-aging formulation (by the inclusion of polyphenols from, for example, grape seed oil, and effective amounts of calcium, among others, including effective amounts of antioxidants), as a composition to repair dental enamel, among others, as otherwise described herein.

For example, in the case of anti-plaque or anti-microbial formulations, the inclusion of colostrum protein as an effective agent is important. Colostrum may be included with one or more proteins selected from the group consisting of lysozyme and lactoperoxidase, preferably in combination with both lysozyme and lactoperoxidase, optionally, in the presence of lactoferrine. Other optional proteins which may be included in this aspect of the present invention are selected from the group consisting of vegetable peroxidase, dextranase, mutanase, cellulase, papain and bromelin. In this aspect of the present invention, dental care compositions comprising an effective amount of colostrum in combination with other additives typically used to provide dental care compositions as toothpaste, gel, paste, pill, tablet, dry tablet, spray, solution or lozenge, soaked napkin or dental floss, among others, are contemplated by this aspect of the invention. Thus, an effective amount of colostrum and optionally, one or more protein as otherwise described above, may be formulated with at least one other additive typically used to produce dental care compositions selected from the group consisting of solvents, diluents, thickeners, gelling agents, abrasives, flavoring agents, coloring agents, anti-cavity agents, anti-microbial agents, anti-tartar agents, film-forming agents, dental enamel mineralizers, dental enamel protectors and whiteners, sweeteners, agents to improve appearance, anti-halitosis agents, anti-inflammatory agents, anti-sensitivity agents, coloring agents, sweeteners, especially non-nutritive sweeteners, nutritive cleaners and surfactants, anti-aging compositions and mixtures thereof . In dental care compositions according to the present invention, the amount of colostrum included is an effective amount, which generally includes an amount ranging from about 0.5% to about 7.5% by weight, more preferably about 1% to about 5% by weight. Preferred compositions comprise a combination of an effective amount of colostrum in combination with an effective amount of lactoperoxidase and/or lysozyme. In such compositions, lactoperoxidase comprises about 0.01% to about 2% by weight of the final composition and lysozyme comprises about 0.05% to about 3% by weight. In this aspect of the present invention, the weight of colostrum to either lactoperoxidase or lysozyme preferably falls within the range of about 1:3 to about 100:1, more preferably about 1:1 to about 10:1, with a most preferred range of about 1:1 to about 3:1. Other optional proteins for use in this aspect of the present invention include, for example, lactoferrin, in amounts ranging from about 1% to about 3%, along with one or more other proteins selected from the group consisting of vegetable peroxidase, dextranase, mutanase, cellulase, invertase, papain, bromelin and mixtures thereof, all used in effective amounts ranging from about 0.01% to about 5% by weight.

In accordance with the preferred mode of implementation of the present invention, the active agents which comprise the present invention and have been set or absorbed by the base material are chosen, together or separately, from among anti-cavity agents such as fluoride salts, anti-microbial agents to fight against bacterial flora developing in dental plaque, anti-tartar agents and component elements of the teeth (such as calcium or other nutrients), among other agents as otherwise described in the present invention.

Other aspects of the present invention relate to specific dental care compositions which may be formulated to emphasize a particular effect. For example, dental care compositions according to the present invention may be formulated to emphasize anti-tartar activity by including effective amounts of anti-tartar components such as papain, zinc chloride, zinc citrate, the phosphate salts, carboxylic acids and their water soluble salts, etc. Anti-halitosis activity may be conferred on compositions according to the present invention by including effective amounts of an anti-halitosis agent such as cysteine, methionine, oil of parsley, oil of mint, oil of eucalyptus or chlorophyll, among others. Compositions according to the present invention may be formulated as agents to treat sensitive teeth by including sodium or potassium nitrate and/or strontium salts, such as strontium chloride, etc., whereas anti-plaque and anti-microbial activity may be conferred to compositions according to the present invention by including colostrum and/or one or more protein selected from the group consisting of lactoperoxidase, lysozyme, lactoferrin, other peroxidases, especially including vegetable peroxidase, dextranase, mutanase, cellulase, invertase, papain, bromelin and mixtures thereof. Whitening compositions which also protect the teeth may be formulated by including effective amounts of a whitening agent such as peroxide, sodium hypochloride and lichen, among others, as well as sodium bicarbonate and trichlosan. Mineralizing/remineralizing compositions may be formulated by including effective amounts of mineral and organic apatite and hydroxyapatite salts.

In a preferred embodiment, the present invention relates to a new product as a dental composition for dental hygiene available in the form of a single-dose ingestible tablet with a matrix that gels when it comes into contact with water or saliva and permits the active ingredients contained within to have an extended action on the teeth and gums. Existing hygienic products have a short time of action in the mouth and must then be spit out again; the present invention remedies this problem by remaining in contact with all the teeth as a hydrocolloid matrix tablet that gels when it comes into contact with saliva, or as semi-hydrated single dose, and contains the specific lipophilic and/or hydrophilic main ingredients to protect and strengthen the teeth. This invention, for human or animal application, is available in a single-dose pill, tablet or ingestible base material that, depending on the appropriate compound, may be swallowed with no deleterious effect and which continues to act as it remains in the saliva.

Particularly preferred compounds according to the present invention are characterized in that, in addition to the gelling matrix materials, the composition contains at least one agent selected from the group consisting of a fluoridated anti-cavity component, an anti-tartar agent, a film-forming agent, a sensitive teeth agent, a chemical anti-microbial agent, a biological anti-microbial agent, an anti-inflammatory agent, a whitening agent, a cleaning agent, an anti-halitosis agent, a polyalcohol agent, a vitamin agent, an abrasive agent, coloring agent, a flavoring agent, a biochemical anti-sugar agent, an agent to protect tooth enamel, a mineralizing agent, an anti-plaque agent and mixtures thereof.

Advantageously, the active elements can also be combined with flavor agents or flavorings, contributing to the appeal and significance of the product, especially for animals or children.

In preferred embodiments according to the present invention, the active agents for use in the present invention may consist essentially of one or more of the following agents, adapted, where applicable, for use in an oral dental hygiene composition:

As anti-cavity agents, fluoridated components long known for their preventive effect, such as sodium fluoride, sodium monofluorophosphate, potassium sodium fluoride, tin fluoride, organic fluorides such as long-chain amine fluorides, for example, oleylamine fluoride or ethanolamine fluorine hydrate, cetylamine hydrofluoride or cetylamine fluoride, bis(hydroxyethyl)-aminopropyl-N-hydroxyethyl-octadecylamine dihydrofluoride, fluorine silicate, for example potassium or sodium hexafluorosilicate, fluorophosphates such as ammonium sodium (also potassium, magnesium or calcium) monofluorophosphate, and/or fluorozirconates, for example sodium (also potassium or tin) fluorozirconate. Plants or their extracts rich in fluorine, such as green tea or spirulina algae or litho-tame may also be used in the present compositions. The amount of fluorine to be included in certain preferred compositions according to the present invention ranges from about 0.01 and 5% of fluorine ion per dose, separately or combined, by weight of the final composition.

As chemical anti-microbial agents, bromochloroprene, between 0.01 and 1%, in relation to the product of the invention. Other appropriate chemical antimicrobial substances that can prevent or at least reduce the formation of dental plaque caused by bacteria are, in particular, the 1,6-bis(p-chlorophenyldiguanido)hexane, or chlorohexadine, in the form of water-soluble salts such as digluconate, diacetate, dilactate, dichlorohydrate, as well as 1,6-di(2-ethylhexyldiguanido)hexane or hexetidine, also in the form of water-soluble salts, the 1.6-di(2-benzyldiguanido)-hexane, hexamidine, p-chlorophenyl diguanide, Nn-(4-chlorobenzyl)-N5-(2.4-dichlorobenzyl)oliguanide, also in the form of water-soluble salts, biguanides, as well as components that include quaternary ammonium, cetylbenzyl dimethylammonium chloride, and urea, are also appropriate to use as a preferred anti-microbial substance, to be processed at between about 0.01 and 2.5% or more, by weight of the final composition.

As anti-tartar agent of active zinc to prevent the formation of tartar, for example, zinc chloride, zinc phenyl-alanine and zinc citrate, for example, in the form of a trihydrate, in a proportion of between about 0.01 and 3% in weight, calculated based on Zn, in relation to the product of the invention, sodium benzoate at between 0.01 and 1.5% in relation to the product of the invention. The various phosphoric acids and their water-soluble salts, for example ethane-0.1-hydroxy- 1,1 -diphosphoric acid, ethylene diaminotetraphosphoric acid, hexamethylene diaminotetraphosphoric acid, complex-forming carboxylic polyacids, particularly citric acid and tartaric acid, as well as their water-soluble salts, with a proportion of between about 0.05 and 5% by weight of the final composition.

As immune agents, colostrum rich in non-specific IGE against germs in the oral cavity, with a proportion of between about 0.1 and 5% by weight of the final composition.

As biological anti-bacterial agent lysozyme or muccopolyssacharidase made from eggs, lactoperoxydase made from milk, vegetable peroxydase, or lactoferrine made from milk, with a proportion of between about 0.05 and 5% by weight of the final composition. A particularly preferred biological anti-bacterial agent which may be used alone or preferably in combination with the gelling agent is colostrum, preferably bovine colostrum.

As biochemical anti-plaque agent, dextranase, mutanase, cellulase, papain, bromelin, with a proportion of between about 0.05 and 5% by weight of the final composition.

As a film-forming agent to protect against tar from tobacco and other tannins, PEG, Vaseline jelly, liquid paraffin, dimethicone, magnesium stearate, or stearic acid, with a proportion of between about 0.05 and 5% by weight of the final composition.

As mineralizer of dental enamel, calcium in any form of mineral and organic apatite and hydroxyapatite salts; the proportion can be between about 0.05 and 5% by weight of the final composition.

As protector of dental enamel and whitener, sodium bicarbonate, triclosan, lichen, peroxide and derivatives, or sodium hypochloride and its salts, with a proportion of between about 0.01 and 5% by weight of the final composition.

As biochemical sugar-free agent, inverted sugar, dehydrogenase sorbitol, glucose oxydase, with a proportion of between about 0.05 and 5% by weight of the final composition.

As flavoring, menthol, eucalyptus, strawberry, lemon, cherry, bubble gum or other flavoring, with a proportion of between about 0.01 and 1.5% by weight of the final composition.

For appearance, fish oil, yeast, liver powder, or glutamate, with a proportion of between about 0.1 and 10% by weight of the final composition.

As a fine abrasive, micronized silica, calcium carbonate, sodium glycerophosphate and/or dicalcic of inorganic phosphates such as sodium trimetaphosphate and organic phosphates, particularly phosphoric esters of polyalcohols, such as sodium or calcium glycerophosphate or sucrose calcium phosphate, with a proportion of between about 0.05 and 5% by weight of the final composition.

As sweeteners, sodium, aspartame, acetosufan, glyceryzinic saccharinates, with a proportion of between about 0.05 and 5% by weight of the final composition.

As vitamins, vitamins A, D, E, with a proportion of between about 0.05 and 5% by weight of the final composition.

As non-cariogenic polyalcohol, lactitol, sorbitol, xylitol, with a proportion of between about 1 and 85% by weight of the final composition.

As halitosis treatment, or against bad breath, cysteine, N-acetyl cysteine, methionine, glutathione, oil of parsley, essential oil of mint, essential oil of eucalyptus, or chlorophyll, with a proportion of between about 0.05 and 5% by weight of the final composition.

As an anti-inflammatory for the gums, 18 beta glycerhetinic acid or enoxolone, salicylic acid and its salts, azulene, ginkgo biloba, or witch hazel, with a proportion of between about 0.01 and 5% by weight of the final composition.

As an ingredient for sensitive teeth, strontium chloride and sodium or potassium nitrate, with a proportion of between about 0.01 and 1.5% by weight of the final composition.

As coloring agent, red, blue or green food coloring, with a proportion of between 0.01 and 0.1% by weight of the final composition.

As nutritive cleaner, polysorbates or sorbitan derivatives, for example the Tween™ and Span™ surfactants, with a proportion of between about 1 and 10% by weight of the final composition.

Of course, the present invention is not limited to the specially considered examples shown above; on the contrary, it encompasses all kinds of variations, particularly with respect to the nature of the active elements incorporated into the base material, as well as to their proportions relative to these elements.

In particular, in accordance with this invention, tablets or single doses of concentrated partially hydrated gel may be prepared, using a weight adapted to the size of the human being or the animal for which they are destined.

With respect to another characteristic of the product of the invention, the base material is made up of:

As base material or lipophilic matrix, having the property of absorbing liquid lipophilic substances, it gels in contact with water and forms a biofixating network, among these substances there are acrylic or polyacrylate derivatives, micronized silica, aerosyl type, or Polyvinyl pyrolidone (PVP), with a proportion of between about 0.05 and 15% by weight of the composition according to the present invention;

As base material or hydrophilic matrix, a water soluble cellulose polymer, for example, carboxymethylcellulose, or simply CMC, is a derivative of cellulose; it is a vegetable polymer and is therefore commonly found in nature. It is, notably, the main component element in wood, cotton, straw, sugarcane and grasses. Cellulose is a polysaccharide made up of groups of glucose, and the formula may be written as follows: natural cellulose has a molecular weight of between 500,000 and 1,500,000. The chemical reactions that provide CMC are sometimes accompanied by significant deterioration, which decreases the molecular weight back to lower values of between 80,000 and 800,000.

Cellulose is not water-soluble by nature. It is only endowed with this property following chemical reactions that provide CMC, on the one hand, and on the other, salts, as by-products. Raw CMC loses its salts when it is washed in water and alcohol, after which it provides a particularly pure product.

CMC dissolves in either hot or cold water. Given its affinity for water, it must only be added slowly and with constant mixing in order to avoid the formation of lumps. It is generally mixed with other pulverulent products to facilitate its dissolution. Derivatives such as hydroxypropyl cellulose, hydroxymethyl cellulose, hydroxyethyl cellulose, methylethyl cellulose, hydroxypropylmethyl cellulose, and methyl cellulose are also used in this invention for their water-soluble properties, and also to achieve a viscous gel very rapidly; the proportion can be between about 0.1 and 25%, more preferably about 1% to about 15% by weight by weight of the final composition.

Beta cyclodextrine can also be incorporated as an optional gelling agent in combination with the water soluble cellulose derivative, preferably CMC, with a proportion of between about I and 10% by weight of the final composition.

Carob gum is extracted from the endosperm of the grain of the carob tree. It is easily soluble in hot water and must be heated to achieve its optimum viscosity. At a concentration of 1% in water, the carob gum shows a viscosity of between about 1,500 to 3,500 cps. It shows significant synergies when combined with other hydrocolloids. The proportion can be between about 0.1 and 15% by weight of the final composition.

Guar gum, like carob gum, is a lactomannan. It hydrates very rapidly in saliva or cold water to form a high-viscosity gel. In an aqueous solution of 1%, the viscosity can vary from 2,500 to 8,000 cps, depending on the quality. Derivatives can also be used such as guar hydroxy; the proportion of the guar gum or related derivative can be between about 0.1 and 15% by weight of the final composition.

Karaya gum comes from the dried exudation of the “Sterculia Urens” tree, which is widespread in India. Karaya gum forms rapidly in viscous colloidal solutions, but does not dissolve easily in water. In a solution of 1%, the average viscosity is about 3,000 to 3,500 cps. Combined with other hydrocolloids, it shows a significant propensity for forming a gel as a linking and adhesive agent for the active ingredients by physical or electrostatic force; the proportion can be between about 0.1 and 10% by weight of the final composition, noting that the amount of karaya gum included in the final composition will depend upon the amount and type of the other gums, notably the water soluble gums used in the final product.

The dry or partially-hydrated gelled concentrate form of this combination of hydrocolloid matrix permits an animal to chew or to lick as it pleases, by triggering the release of the active ingredients.

The present compositions are prepared using standard formulary practice in the food, pharmaceutical and dental formulation arts. In general, compositions according to the present invention which are prepared as powders, tablets or the like and which gel after being exposed to an aqueous solution such as saliva, water or juice in the mouth of the patient or subject are prepared by homogenizing a mixture of components, in solid, liquid or gel form depending upon the final dosage form and then finalizing the dosage form using routine practices which are well known in the art to produce oral dosage forms such as toothpastes, gels, paste pills, sprays, solutions, lozenges, gums, soaked napkins, dental floss and any other form which may be administered orally.

The following examples are provided to illustrate the present invention:

### EXAMPLES

Example 1
Chewable Dentifrice

| Component | Weight Percent of Composition |
|---|---|
| BROMOCHLOROPHENE | from 0.01 to 1% |
| ENOXOLONE | from 0.1% to 3% |
| SODIUM BICARBONATE 40 | from 1% to 5% |
| MICRONIZED SILICA | from 1% to 5% |
| SORBITOL Direct compressible | from 45% to 60% |
| XYLITOL Direct compressible | from 5% to 40% |
| LIVER POWDER | from 1% to 15% |
| METHIONINE/CYSTEINE | from 0.1% to 3% |
| COLORING 5 | 0.001 to 0.1% |
| PAPAIN/BROMELIN | from 0.01 to 1% |
| GLUCOSE OXIDASE/LACTOPEROXYDASE | from 0.01 to 1% |
| AMYLOGLUCOSIDASE/INVERTASE | from 0.01 to 1% |
| LYSOZYME/LACTOFERRIN | from 0.01 to 1% |

The above components may be mixed to homogeneity and then pressed into tablets for administration.

Example 2
Digestible Dentifrice Tablet

| Component | Weight Percent of Composition |
|---|---|
| AEROSIL 300 (Silica) | from 0.1% to 3% |
| ASPARTAME/SACCHARIN | from 0.1% to 1% |
| SILICA HYDRATE | from 0.5 to 5% |
| DTMETHICONE/VASELNE JELLY | from 0.01% to 1% |
| Essential oil of Mint/Eucalyptus or Strawberry | from 1 to 5% |
| ALGAE POWDER Fluoridated in NaF | from 2% to 5% |
| SODIUM BENZOATE 20 | from 0.1 to 1% |
| MAGNESIUM STEARATE/STEARIC ACID | from 2.0 to 5% |
| Menthol crystals | from 0.1 to 1% |
| ZINC CITRATE | from 0.1% to 3% |
| Lactomannan, alginate, carrhaguenate | from 1% to 15% |
| SORBITAM 80 | from 0.01 to 1% |
| BROMOCHLOROPHENE/CHLORHEXIDINE | from 0.01 to 1% |
| VITAMINS A, D, E | from 0.01 to 2% |
| CALCIUM/TRICLOSAN PEROXIDE | from 0.1% to 5% |
| ENOXOLONE | from 0.1% to 3% |
| CALCIUM SALT 30 | from 0.1% to 5% |
| SODIUM BICARBONATE | from 1% to 5% |
| MICRONIZED SILICA | from 1% to 5% |
| SORBITOL Direct compressible | from 45% to 60% |
| XYLITOL Direct compressible | from 5% to 40% |
| METHIONINE/CYSTEINIE | from 0.1% to 3% |
| COLORING | 0.00 1 to 0.1% |
| PAPAIN/BROMELIN | from 0.01 to 1% |
| GLUCOSE OXYDASE/LACTOPEROXYDASE | from 0.01 to 1% |
| AMYLOGLUCOSIDASE/INVERTASE | from 0.01 to 1% |
| LYSOZYME/LACTOFERRINE | from 0.01 to 1% |

The above components may be mixed to homogeneity and then pressed into tablets for oral delivery.

Example 3
Single Dose Semi-Solid Gel

| | |
|---|---|
| AEROSIL 300 | from about 0.1% to 3% |
| ASPARTAME/SACCHARINE | from about 0.1 to 1% |
| 45 SILICA HYDRATE | from about 0.5 to 5% |
| DIMETHICONE/VASELINE JELLY | from about 0.01% to 1% |
| Essential oil of Mint/Eucalyptus, Dose 2% | from about 1 to 5% |
| ALGAE POWDER Fluoridated NaF | from about 2% to 5% |
| SODIUM BENZOATE | from about 0.1 to 1% |
| 50 MAGNESIUM STEARATE/STEARIC ACID | from about 2.0 to 5% |
| Menthol crystals | from about 0.1% to 1% |
| ZINC CITRATE | from about 0.1% to 3% |
| Lactomannan, alginate, carrhaguenate | from about 1% to 15% |
| SORBITAM 80 | from about 0.01 to 1% |
| BROMOCHLOROPHENE/CHLOROHEXIDINE | from about 0.01 to 1% |
| VITAMINS A, D, E | from about 0.01 to 2% |
| 5 CALCIUM PEROXIDE | from about 0.1% to 5% |
| ENOXOLONE | from about 0.1% to 3% |
| CALCIUM SALT | from about 0.1% to 5% |
| SODIUM BICARBONATE | from about 1% to 5% |
| MICRONIZED SILICA | from about 1% to 5% |
| 10 GLYCEROL/PVP | from about 5% to 10% |
| XYLITOL Direct Compressible | from about 5% to 10% |

PURIFIED WATER QSP VISCOSITY 10,000 CPS to 30,000 CPS QSP

| | |
|---|---|
| LIVER POWDER | from about 1% to 15% |
| METHIONINE/CYSTEINE | from about 0.1% to 3% |
| COLORING 15 | about 0.001 to 0.1% |
| PAPAIN/BROMELIN | from about 0.01 to 1% |
| GLUCOSE OXYDASE/LACTOPEROXYDASE | from about 0.01 to 1% |
| AMYLOGLUCOSIDASE/INVERTASE | from about 0.01 to 1% |
| LYSOZYME/LACTOFERRIN | from about 0.01 to 1% |

This product is a semi-solid gel which can be delivered to the oral cavity without further gelling in the mouth through action of saliva.

Example 4
Digestible Dentifrice

| | |
|---|---|
| AEROSIL 300 | from about 0.1% to 3% |
| ASPARTAME/SACCHARIN | from about 0.1% to 1% |
| SILICA HYDRATE | from about 0.5% to 5% |
| DIMETHICONE/VASELINE JELLY | from about 0.01% to 1% |
| Essential oil of Mint/Eucalyptus, Dose 2% | from about 1 to 5% |
| ALGAE POWDER, fluoridated in NaF | from about 2% to 5% |
| SODIUM BENZOATE | from about 0.1 to 1% |
| STEARATE OF MAGNESIUM/STEARIC ACID | from about 2.0 to 5% |
| Menthol crystals | from about 0.1 to 1% |
| ZINC CITRATE | from about 0.1% to 3% |
| Lactomannan, alginate, carrhaguenate | from about 1% to 15% |
| SORBITAM 80 | from about 0.01 to 1% |
| BROMOCHLOROPHENE/CHLOROHEXIDINE | from about 0.01 to 1% |
| VITAMINS A, D, E | from about 0.01 to 2% |
| CALCIUM PEROXIDE | from about 0.1% to 5% |
| ENOXOLONE | from about 0.1% to 3% |
| CALCIUM SALT | from about 0.1% to 5% |
| SODIUM BICARBONATE | from about 1% to 5% |
| MICRONIZED SILICA | from about 1% to 5% |
| GLYCEROL/PVP | from about 5% to 15% |
| XYLITOL Direct Compressible | from about 5% to 40% |
| LIVER POWDER | from about 1% to 15% |
| METHIONINE/CYSTEINE | from about 0.1% to 3% |
| COLORING | about 0.001 to 0.1% |
| PAPAIN/BROMELIN | from about 0.01 to 1% |
| GLUCOSE OXYDASE/LACTO-PEROXYDASE | from about 0.01 to 1% |
| AMYLOGLUCOSIDASE/INVERTASE | from about 0.01 to 1% |
| LYSOZYME/LACTOFERRIN | from about 0.01 to 1% |

This composition may be homogenized and formed into a tablet for oral delivery.

It is to be understood by those skilled in the art that the foregoing description and examples are illustrative of practicing the present invention, but are in no way limiting. Variations of the detail presented herein may be made without departing from the spirit and scope of the present invention as defined by the following claims.

1. A composition for oral hygiene in the form of an ingestible dental care product for human and animal use wherein said dental care product is a composition comprising, in single dosage form, an effective amount of silica and optionally at least one ingestible hydrophilic gelling agent, further in combination with at least one active agent for the treatment of teeth or gums in a patient, said composition being delivered in the form of a gel or in a form which gels in the mouth after exposure to an aqueous solution to allow formation of a film from said composition on said teeth or gums of said patient, said composition allowing said active agent within said composition to be retained in the oral cavity for long-term delivery and action.

2. The composition according to claim 1, formulated in the form of a toothpaste, gel, paste, pill, tablet, dry tablet, spray, solution or lozenge, soaked napkin, dental floss or any other prepared form applicable to ingestible dental hygiene products in a single-dose form dosage form.

3. The composition according to claim 1, wherein said composition comprises silica and a hydrophilic gelling agent, said hydrophilic gelling agent being selected from the group consisting of carboxymethyl cellulose, methyl cellulose, methylethyl cellulose, hydroxymethyl cellulose, hydroxyethyl cellulose, hydroxypropyl cellulose, hydroxypropylmethyl cellulose, guar gum, hydroxy guar gum, carob gum, karaya gum, algin, carageenan, and mixtures thereof, said silica and said hydrophilic gelling agent being included in said composition in a weight ratio of silica to hydrophilic gelling agent ranging from about 1:1 to about 1:6.

4. The composition according to claim 1 further comprising a polyol diluent selected from the group consisting of sorbitol, xylitol, mannitol, maltitol, lactitol, glycerol (glycerine) and propylene glycol in an effective amount ranging from about 0.1% to about 85% by weight.

5. The composition according to claim 1 wherein said active is selected from the group consisting of anti-cavity agents, anti-microbial agents, anti-tartar agents, anti-bacterial agents, anti-plaque agents, film-forming agents, mineralizers, teeth whiteners, vitamins, anti-sensitivity agents, anti-halitosis agents, anti-inflammatory agents, nutritive cleaners and anti-aging compounds.

6. The composition according to claim 5 wherein said active agent is selected from the group consisting of anti-cavity agents, anti-microbial agents, anti-plaque agents, mineralizers, teeth whiteners, anti-sensitivity agents and anti-halitosis agents.

7. The composition according to claim 3 comprising at least one lipophilic active agent and at least one hydrophilic active agent.

8. The composition according to claim 1 wherein said active agent is colostrum or a mixture of colostrum and a protein selected from the group consisting of lysozyme, lactoperoxidase, lactoferrin and mixtures, thereof.

9. The composition according to claim 1 wherein said active agent is selected from the group consisting of colostrum, lysozyme, lactoperoxidase, lactoferrin, dextranase, mutanase, cellulase, amylo-glucosidase, papain, bromelin and mixtures thereof.

10. The composition according to claim 1 wherein said hydrophilic gelling agent is selected from the group consisting of a lactomannan, algin, carageenan and mixtures thereof.

11. The composition according to claim 1 wherein said hydrophilic gelling agent is selected from the group consisting of carboxymethyl cellulose, guar gum, carob gum, karaya gum, algin, carageenan and mixtures thereof.

12. The composition according to claim 1 wherein said anti-cavity agent is selected from the group consisting of sodium fluoride, potassium fluoride, sodium monofluorophosphate, potassium sodium fluoride, tin fluoride, fluorine silicate, organic fluorides, fluorozirconates, green tea, spirulina algae, lithotame and mixtures, thereof; said anti-microbial agent is selected from the group consisting of bromochloroprene, chlorohexidine and its pharmaceutically acceptable salts, thereof, hexetidine and its pharmaceutically acceptable salts, 1,6-di(2-benzyldiguanido)-hexane, hexamidine, p-chlorophenyl diguanide, Nn-(4-chlorobenzyl)-N5-2,4-dichlorobenzyl)oliguanide and its pharmaceutically acceptable salts, biguanides, and mixtures thereof; said anti-tartar agent is selected from the group consisting of zinc chloride, zinc phenylalanine, zinc citrate, ethane-1-hydroxy-1,1-diphosphoric acid, ethylene diaminotetraphosphoric acid, hexamethylene diaminotetraphosphoric acid, citric acid and its water soluble salts thereof, tartaric acid and its water soluble salts thereof, and mixtures thereof; said anti-bacterial agent is selected from the group consisting of lysozyme, mucopolysacharidase, lactoperoxidase, vegetable peroxidase, lactoferrin and mixtures thereof; anti-plaque agents selected from the group consisting of dextranase, mutanase, cellulase, papain, bromelin and mixtures thereof; said mineralizer is selected from the group consisting of calcium apatite, calcium hydroxyapatite and mixtures, thereof; said teeth whitener is selected from the group consisting of sodium peroxide, potassium peroxide, sodium hypochloride, potassium hypochloride and mixtures thereof; said vitamin is selected from the group consisting of vitamins A, D, E and mixtures thereof; said anti-sensitivity agent is selected from the group consisting of sodium nitrate, potassium nitrate, strontium chloride and mixtures thereof; said anti-halitosis agent is selected from the group consisting of cysteine, N-acetyl cysteine, methionine, glutathione, oil of parsley, essential oil of mint, essential oil of eucalyptus, chlorophyll and mixtures thereof; said anti-inflammatory agent is selected from enoxolone, 18-beta-glycerhetinic acid, salicylic acid and its pharmaceutically acceptable salts, azulene, ginkgo biloba, witch hazel and mixtures thereof; and said nutritive cleaner is selected from the group consisting of a polysorbate or sorbitan cleaner.

13. The composition according to claim 1 further comprising a lipophilic gelling agent selected from the group consisting of an acrylic derivative, a polyacrylate derivative, polyvinyl pyrrolidone and mixtures thereof.

14. The composition according to claim 1, wherein said active agent is a chemical anti-microbial agent selected from the group consisting of chlorohexidine and/or bromochlorophene.

15. The composition according to claim 1 wherein said active agent is an anti-tartar agent selected from the group consisting of a zinc salt or phosphoric acid salt.

16. The composition according to claim 1, where in said active agent is a protective agent for tooth enamel selected from the group consisting of sodium bicarbonate, triclosan, lichen, and mixtures thereof.

17. The composition according to claim 1, wherein said active agent is a biochemical anti-sugar agent with enzymatic activity selected from the group consisting of invertase, glucose oxidase and mixtures thereof.

18. The composition according to claim 1 further including as a flavoring agent selected from the group consisting of a natural essential plant oil or a synthetic oil.

19. The composition according to claim I wherein said active agent is an anti-aging compound selected from the group consisting of polyphenols, grape seed oil, calcium, antioxidants and mixtures thereof.

20. A dental care composition exhibiting anti-aging activity, said composition comprising an effective amount of at least one agent selected from the group consisting of polyphenols or grape seed oil, said composition further comprising at least one additional additive selected from the group consisting of solvents, diluents, thickeners, gelling agents, abrasives, flavoring agents, coloring agents, anti-cavity agents, anti-microbial agents, anti-tartar agents, film-forming agents, dental enamel mineralizers, dental enamel protectors and whiteners, sweeteners, agents to improve appearance, anti-halitosis agents, anti-inflammatory agents, anti-sensitivity agents, coloring agents, sweeteners, nutritive cleaners and surfactants and mixtures thereof, said composition optionally further comprising at least one additional agent selected from the group consisting of calcium, antioxidants and mixtures thereof.

21. A dental care composition exhibiting anti-plaque activity, said composition comprising an effective amount of colostrum alone or in combination with an effective amount of an additional protein selected from the group consisting of lysozyme, lactoperoxidase and mixtures thereof and further comprising at least one additional additive selected from the group consisting of solvents, diluents, thickeners, gelling agents, abrasives, flavoring agents, coloring agents, anti-cavity agents, anti-microbial agents, anti-tartar agents, film-forming agents, dental enamel mineralizers, dental enamel protectors and whiteners, sweeteners, agents to improve appearance, anti-halitosis agents, anti-inflammatory agents, anti-sensitivity agents, coloring agents, sweeteners, nutritive cleaners and surfactants, anti-aging compositions and mixtures thereof.

22. The composition according to claim 21 comprising an effective amount of colostrum and a mixture of lysozyme and lactoperoxidase, wherein the weight ratio of colostrum to either lysozyme or lactoperoxidase ranges from about 1:3 to about 100:1.

23. The composition according to claim 21 comprising an effective amount of colostrum and a mixture of lysozyme and lactoperoxidase, wherein the weight ratio of colostrum to either lysozyme or lactoperoxidase ranges from about 1:1 to about 10:1.

24. The composition according to claim 21 comprising an effective amount of colostrum and a mixture of lysozyme and lactoperoxidase, wherein the weight ratio of colostrum to either lysozyme or lactoperoxidase ranges from about 1:1 to about 10:1.

25. The composition according to claim 21 further comprising an effective amount of lactoferrin.

26. The composition according to claim 22 further comprising an effective amount of lactoferrin.

27. The composition according to claim 23 further comprising an effective amount of lactoferrin.

28. The composition according to claim 24 further comprising an effective amount of lactoferrin.

29. The composition according to claim 22 wherein said colostrum comprises about 0.5% to about 7.5% by weight of said composition, said lysozyme comprises about 0.05% to about 3.0% by weight of said composition and said lactoperoxidase comprises about 0.01% to about 2.0% by weight of said composition.

30. The composition according to claim 25 wherein said colostrum comprises about 0.5% to about 7.5% by weight of said composition, said lysozyme comprises about 0.05% to about 3.0% by weight of said composition, said lactoperoxidase comprises about 0.01% to about 2.0% by weight of said composition and said lactoferrin comprises about 15 to about 3% by weight of said composition.

31. The composition according to claim 26 wherein said colostrum comprises about 0.5% to about 7.5% by weight of said composition, said lysozyme comprises about 0.05% to about 3.0% by weight of said composition, said lactoperoxidase comprises about 0.01% to about 2.0% by weight of said composition and said lactoferrin comprises about 15 to about 3% by weight of said composition.

32. The composition according to claim 27 wherein said colostrum comprises about 0.5% to about 7.5% by weight of said composition, said lysozyme comprises about 0.05% to about 3.0% by weight of said composition, said lactoperoxidase comprises about 0.01% to about 2.0% by weight of said composition and said lactoferrin comprises about 15 to about 3% by weight of said composition.

33. The composition according to claim 21 further comprising an effective amount of at least one additional protein selected from the group consisting of dextranase, mutanase, cellulase, amylo-glucosidase, papain, bromelin and mixtures thereof.

34. The composition according to claim 22 further comprising an effective amount of at least one additional protein selected from the group consisting of dextranase, mutanase, cellulase, amylo-glucosidase, papain, bromelin and mixtures thereof.

35. The composition according to claim 25 further comprising an effective amount of at least one additional protein selected from the group consisting of dextranase, mutanase, cellulase, amylo-glucosidase, papain, bromelin and mixtures thereof.

36. A method of treating the teeth or gums of a patient, said method comprising the steps of
a. administering the composition according to claim 1 to the oral cavity of the patient;
b. depositing said composition in a manner consistent with the deposition of said composition onto said teeth or gums of said patient; and
c. ingesting said composition after said depositing step.

37. A method of treating the teeth or gums of a patient, said method comprising the steps of
a. administering the composition according to claim 5 to the oral cavity of the patient;
b. depositing said composition in a manner consistent with the deposition of said composition onto said teeth or gums of said patient; and
c. ingesting said composition after said depositing step.

38. A method of preventing cavities in a patient, said method comprising the steps of
a. administering the composition according to claim 21 to the oral cavity of the patient;
b. depositing said composition in a manner consistent with the deposition of said composition onto said teeth or gums of said patient; and
d. ingesting said composition after said depositing step.

39. The composition according to claim 21, formulated in the form of a toothpaste, gel, paste, pill, tablet, dry tablet, spray, solution or lozenge, soaked napkin or dental floss.

40. The composition according to claim 23, formulated in the form of a toothpaste, gel, paste, pill, tablet, dry tablet, spray, solution or lozenge, soaked napkin or dental floss.

41. The composition according to claim 24, formulated in the form of a toothpaste, gel, paste, pill, tablet, dry tablet, spray, solution or lozenge, soaked napkin or dental floss.

42. The composition according to claim 25, formulated in the form of a toothpaste, gel, paste, pill, tablet, dry tablet, spray, solution or lozenge, soaked napkin or dental floss.

43. The composition according to claim 35, formulated in the form of a toothpaste, gel, paste, pill, tablet, dry tablet, spray, solution or lozenge, soaked napkin or dental floss.

44. A method of reducing plaque or plaque formation on the teeth or microbial growth on the teeth or in the mouth of a patient, said method comprising exposing the teeth or mouth of said patient to an effective amount of the composition according to claim 21.

45. A method of reducing the likelihood that cavities will form in the teeth of a patient at risk for cavity formation, said method comprising exposing the teeth or mouth of said patient to an effective amount of the composition according to claim 21.

46. A method of reducing plaque or plaque formation on the teeth or microbial growth on the teeth or in the mouth of a patient, said method comprising exposing the teeth or mouth of said patient to an effective amount of the composition according to claim 23.

47. A method of reducing the likelihood that cavities will form in the teeth of a patient at risk for cavity formation, said method comprising exposing the teeth or mouth of said patient to an effective amount of the composition according to claim 23.

48. A method of reducing plaque or plaque formation on the teeth or microbial growth on the teeth or in the mouth of a patient, said method comprising exposing the teeth or mouth of said patient to an effective amount of the composition according to claim 33.

49. A method of reducing the likelihood that cavities will form in the teeth of a patient at risk for cavity formation, said method comprising exposing the teeth or mouth of said patient to an effective amount of the composition according to claim 33.

50. A method of reducing plaque or plaque formation on the teeth or microbial growth on the teeth or in the mouth of a patient, said method comprising exposing the teeth or mouth of said patient to an effective amount of the composition according to claim 35.

51. A method of reducing the likelihood that cavities will form in the teeth of a patient at risk for cavity formation, said method comprising exposing the teeth, gums or mouth of said patient to an effective amount of the composition according to claim 35.

52. A method of treating periodontal disease in a patient or reducing the likelihood that a patient will contract periodontal disease in a patient at risk for said disease, said method comprising exposing the teeth, gums or mouth of a patient to an effective amount of the compound according to claim 21.
